(19) United States
(12) Patent Application Publication
(10) Pub. No.: US 2021/0146025 A1
(43) Pub. Date: May 20, 2021

(54) Methods and Systems for Collecting Whole Blood from a Donor and Maintaining Fluid Volume

(71) Applicant: Fenwal, Inc., Lake Zurich, IL (US)

(72) Inventor: Katherine N. Radwanski, Highland Park, IL (US)

(21) Appl. No.: 17/096,305

(22) Filed: Nov. 12, 2020

Related U.S. Application Data
(60) Provisional application No. 62/935,345, filed on Nov. 14, 2019.

(51) Int. Cl. A61M 1/02; A61M 39/28

(52) U.S. Cl. CPC A61M 1/0209; A61M 39/28; A61M 2202/0429; A61M 2205/75; A61M 2202/0427; A61M 2205/0205

(57) ABSTRACT

Methods and systems for the manual collection of whole blood are disclosed. The methods and systems include delivering a replacement fluid to the donor after or during the course of the blood collection.

### FIELD OF THE DISCLOSURE

The present disclosure is directed to methods and systems for collecting whole blood from a donor and maintaining the fluid volume in such donor. More particularly, the present disclosure is directed to methods for collecting whole blood from a donor and providing a replacement fluid to the donor, and to disposable fluid circuits for performing the method. Even more particularly, the present disclosure is directed to disposable fluid circuits that include a container of replacement fluid or are configured to receive a container of replacement fluid.

### BACKGROUND

Whole blood is made up of various cellular components such as red blood cells, white cells and platelets suspended in its liquid component, plasma. Whole blood is collected from a donor and typically separated into its constituent components (cellular or liquid), and the desired separated component can be administered to a patient in need of that particular component. For example, platelets can be removed from the whole blood of a healthy donor, collected, and later administered to a cancer patient whose ability to “make” platelets has been compromised by chemotherapy or radiation treatment. Similarly, red blood cells can be separated from the whole blood, collected and stored for later transfusion to a patient in need of red blood cells.

The collection of whole blood may be carried out “manually” or in an automated fashion. In manual whole blood collection, blood is withdrawn from the donor through a venipuncture needle into a (plastic) collection container. The needle and the collection container are typically part of a larger disposable fluid circuit that includes one or more plastic satellite containers and plastic tubing defining flow paths that are in openable flow communication with the containers. The satellite containers may be (initially) empty and configured to receive blood components that have been separated from whole blood. Additional containers may include a container of an additive or preservative solution for facilitating the storage of a separated blood component, or otherwise condition the component for further processing or treatment. In any event, the containers, needle and the tubing that connects the same form an integrated, self-contained disposable fluid circuit or kit. Once the desired volume of whole blood has been collected in the collection container, the collected blood is later separated (by, for example, subjecting it to centrifugation) into its components which may then be transferred to the pre-attached satellite containers. In a manual whole blood collection, no blood, blood components, or other solutions are currently returned to the donor.

The automated processing of blood and blood components typically involves using a reusable processing apparatus (“hardware”) and a disposable fluid circuit adapted for mounting or other association with the reusable apparatus. The processing apparatus includes pumps and valves which move fluid and control flow through the circuit under the direction of a programmable controller. As in manual collection, the fluid circuit typically includes (plastic) bags and associated tubing that defines a flow path through the circuit. The disposable fluid circuit may also include one or more separation devices including a separation chamber where the biological fluid/cells can be separated into two or more components, washed or otherwise processed. In automated processing, often referred to as “apheresis,” the donor typically remains connected to the apparatus during processing, collection and separation. Moreover, in apheresis, while certain components are collected, other components may be returned to the donor.

In whole blood collection, whether by a manual process or by automated apheresis, the donor may lose an amount of blood, or experience a decrease in blood volume which can also cause a drop in blood pressure or hypotension. The risk of hypotension can be more easily mitigated in automated apheresis collections by automatically introducing additional fluids to the donor either after or during the procedure while the donor is still connected to the apparatus. For example, some devices, like the Amicus Separator, available from Fresenius Kabi, Lake Zurich, Ill., are programmed to deliver a saline bolus to the donor during the collection if hypotension occurs. Unfortunately, similar measures are absent in manual whole blood collection procedures. Thus, it would be desirable to provide a way to deliver replacement fluids during a manual whole blood collection procedure.

### SUMMARY

In one aspect, disclosed herein is a disposable fluid circuit for the manual collection of whole blood. The fluid circuit includes a primary container for collecting and processing whole blood and one or more secondary containers downstream of and in openable flow communication with the primary container. A donor access device is provided and a donor access line defining a flow path is in flow communication with the donor access device. The fluid circuit may further include a replacement fluid container containing a replacement fluid. A branch member, which is in flow communication with the donor access line includes a first branch in openable flow communication with the primary container through a primary container line and a second branch that is in openable flow communication with the replacement fluid container by a replacement fluid line.

In another aspect, the disposable fluid circuit for the manual collection of whole blood includes a primary container for collecting and processing whole blood and one or more secondary containers in openable flow communication with the primary container, a donor access device, a donor access line defining a flow path in flow communication with the donor access device, and a branch member in flow communication with said donor access line. The branch member includes a first branch in openable flow communication with the primary container through a primary container line and a second branch in openable flow communication with a replacement fluid line. The replacement fluid line includes an access site at or near its terminal end for sterile connection of a replacement fluid container.

In another aspect, disclosed herein is a method for maintaining fluid volume in a donor during a manual collection of whole blood with a disposable fluid circuit of the type described herein. The method includes withdrawing blood from a donor through the donor access device and collecting a selected volume of blood in the primary container. The method further includes closing the primary container line by actuating the re-openable flow controller on the primary container line and opening the replacement fluid line to deliver a selected volume of replacement fluid from a replacement fluid container through said replacement fluid line to said donor through said access device.

In further, more specific aspects of the method, the collection may be paused and, if necessary, a selected amount or bolus of replacement fluid may be delivered to the donor prior to arriving at a selected volume of blood collected in the primary container, or after arriving at a selected volume and said blood collection is collected.

### BRIEF DESCRIPTION OF THE FIGURES

FIG. 1 shows one embodiment of a disposable fluid circuit in accordance with the present disclosure;

FIG. 2 shows an alternative embodiment of a disposable fluid circuit in accordance with the present disclosure; and

FIG. 3 is a flow chart setting forth the steps of the method of maintaining fluid volume in accordance with the present disclosure.

### DETAILED DESCRIPTION OF THE EMBODIMENTS

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter of the invention.

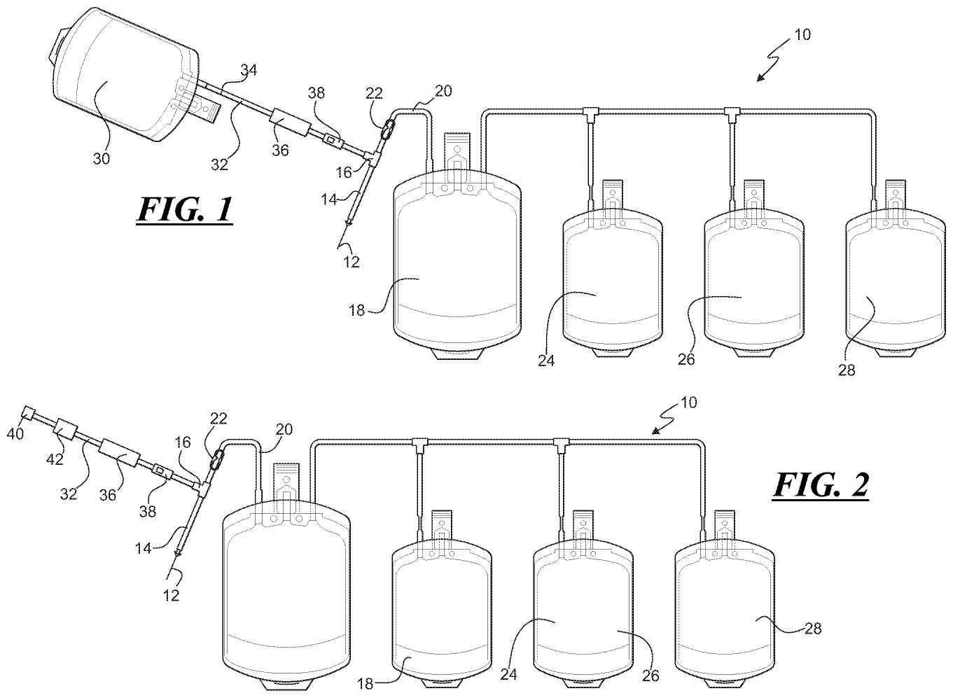

Turning now to the Figures, there is shown in FIG. 1 a disposable fluid circuit in accordance with the present disclosure. As shown in FIG. 1, circuit 10 includes a donor access device 12 for accessing the circulatory system of a donor. Typically, donor access device 12 is a needle or cannula. Access device 12 is in flow communication with donor access line 14 which leads to a branch member 16. Branch member 16 includes a first branch 16a and a second branch 16b. Primary container 18 is in flow communication with first branch member 16a via or through primary container line 20. As further shown in FIG. 1, fluid circuit 10 may include one or more satellite containers 24 and 26 for receiving separated blood components from primary container 18. In addition, fluid circuit 10 may also include an additive solution such as a preservative which may be added to a separated component such as red blood cells or platelets, as the case may be.

Fluid circuit 10 may further include a replacement fluid container 30. Preferably, fluid replacement container 30 is in flow communication with second branch 16b of branch member 16. Fluid replacement container 30 includes a replacement fluid such as saline or other crystalloid or colloid solutions. Typical fill volumes for replacement fluid container may range from 100-500 ml but more preferably may be approximately 250 ml. As shown in FIG. 1, replacement fluid container 30 is in flow communication with branch member 16 and ultimately donor access device 12 by replacement fluid line 32. Replacement fluid line 32 may include a breakable or frangible cannula 34 which prevents flow from replacement container 30 until administration of the replacement fluid is desired. As further shown in FIG. 1, replacement fluid line 32 may also include drip chamber 36 to allow air to escape the liquid and prevent air embolisms from entering the donor's circulatory system and to estimate fluid flow rate.

In accordance with the present disclosure, replacement fluid line may further include a re-openable flow controller or clamp 38. Clamp 38 may preferably be a traditional Robert's type clamp that can be opened and closed or more preferably a roller clamp which likewise can be opened or closed and which can regulate the flow of fluid through replacement container line 32 and back to the donor.

FIG. 2 shows an alternative embodiment of the disposable fluid circuit 10 described above. In this alternative embodiment, the circuit 10 does not include a pre-attached replacement container 30. Rather, the fluid circuit 10 includes an access site 40 at or near the terminal end of replacement fluid line 32. In this embodiment, a replacement fluid container 30 may be attached to disposable fluid circuit 10 at access site 40 at the time of blood collection. Where the replacement container is attached to the access site 40, the circuit 10 may further include a microbial or other filter 42 in the flow path of replacement container line 32 to prevent bacteria or other pathogens from entering the line and being conveyed to the donor. In yet another embodiment, fluid circuit 10 may be as described above except that the microbial or other filter 42 may be housed within the drip chamber 36, as generally depicted by the dashed lines 43 in FIG. 2.

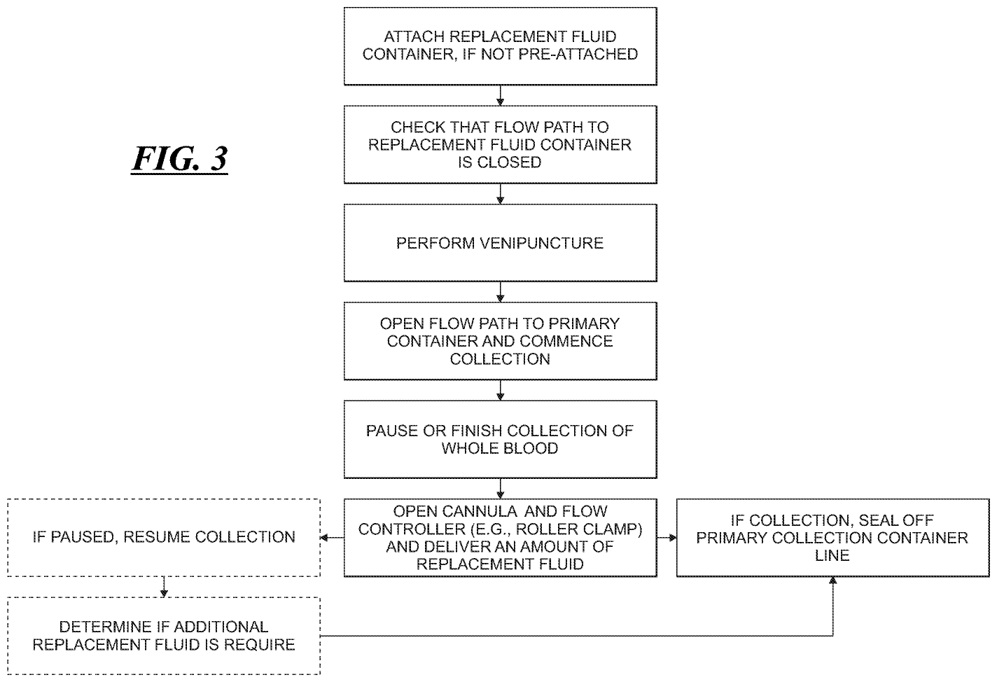

With reference to FIG. 3 and turning now to the method of maintaining the blood volume of a donor using the disposable fluid circuit described above, where the replacement container 30 is pre-attached to circuit 10 or attached at the time of use, the method may commence by ensuring that, the flow controller 38 on replacement fluid line 32 is closed, and then inserting access device 12 into the donor and establishing flow communication with the flow circulatory system. (Of course, if using a circuit that does not have a pre-attached container, prior to making the venipuncture, a container 30 may be attached to circuit 10 at access site 40, as shown in FIG. 3). If primary container line includes the flow controller or clamp 22, such clamp is opened at the time of donation and whole blood is withdrawn from the donor and introduced into primary container 18. During blood collection, flow from replacement fluid container 30 is prevented by breakable cannula of 34. Once collection of the whole blood is completed or paused the operator may seal off (by, for example, heat sealing) primary container line 20 and begin administration of the replacement fluid from replacement fluid container 30. Accordingly, breakable cannula 34 is broken or fractured and reopenable flow controller 22 is placed in the open position to allow replacement fluid to flow through replacement container line 32 and into the donor. The operator may manipulate the roller clamp 28 to control the rate of flow and the amount of flow being returned to the donor. The volume of replacement fluid administered to the donor can be a standard volume, such as 250 mL, or any volume which can be safely reinfused to any donor regardless of their size. Alternatively, the volume of replacement fluid administered to the donor may vary based on the donor's volume of blood collected. This volume administered may be estimated by reference to the gradations on the replacement fluid container 30 or by other means including visual observation, changes in height and the like.

Replacement fluid can be administered to the donor after completion of the collection of whole blood, or can be introduced during the procedure or process of collecting whole blood, if necessary. In that aspect, when the operator recognizes that the donor blood volume or blood pressure has dropped, such that the donor becomes symptomatic (lightheadedness, sweating, pallor, etc.) he or she may pause collection by closing clamp 22 on primary container line 20. At that point, cannula 34 may be broken and clamp, such as roller clamp 38, may be placed in an open position to deliver a selected volume or bolus of replacement fluid to the donor. Collection of whole blood may then be resumed by closing the reopenable clamp or flow controller 38, opening the flow controller 22 on primary container line 20 and resuming the collection. Additional replacement fluid may be added at the end of the collection or at other intervening moments of the blood collection.

It will be understood that the embodiments described above are illustrative of some of the applications of the principles of the present subject matter. Numerous modifications may be made by those skilled in the art without departing from the spirit and scope of the subject matter disclosed herein, including those combinations of features that are individually disclosed or claimed herein. For the reasons, the scope hereof is not limited to the above decryption.

### OTHER EXAMPLES

In addition to the aspects set forth in the above summary, description, and the claims that follow, there are other aspects of the present subject matter which may be embodied separately or together in the methods and systems described and claimed below. These aspects may be employed alone or in combination with other aspects of the subject matter described herein, and the description of these aspects together is not intended to preclude the use of these aspects separately or the claiming of such aspects separately or in different combinations, as set forth in the claims appended hereto.

Aspect 1. A disposable fluid circuit for the manual collection of whole blood including: a primary container for collecting and processing whole blood and one or more secondary containers downstream of and in openable flow communication with the primary container; a donor access device and a donor access line defining a flow path in flow communication with the donor access device; a replacement fluid container containing a replacement fluid; and a branch member in flow communication with the donor access line. The branch member includes a first branch in openable flow communication with the primary container through a primary container line and a second branch in openable flow communication with the replacement fluid container by a replacement fluid line.

Aspect 2. The fluid circuit of Aspect 1 wherein the replacement fluid line includes an openable cannula between the second branch and the replacement fluid container.

Aspect 3. The fluid circuit of Aspect 1 wherein the replacement fluid line also includes a drip chamber positioned downstream of the cannula.

Aspect 4. The fluid circuit of any one of Aspects 1 through 3 wherein the replacement line includes a flow controller on the replacement fluid line.

Aspect 5. The fluid circuit of Aspect 4 wherein the flow controller includes a re-openable clamp.

Aspect 6. The fluid circuit of any one of Aspects 1 through 5 wherein the primary container line includes a re-openable flow controller.

Aspect 7. The fluid circuit of any one of Aspects 1 through 6 wherein the replacement fluid is saline, or a crystalloid or colloid solution.

Aspect 8. A disposable fluid circuit for the manual collection of whole blood including: a primary container for collecting and processing whole blood and one or more secondary containers in openable flow communication with the primary container; a donor access device and a donor access line defining a flow path in flow communication with the donor access device; and a branch member in flow communication with the donor access line wherein the branch member includes a first branch in openable flow communication with said primary container through a primary container line and a second branch in openable flow communication with a replacement fluid line. The replacement fluid line terminates at an access site for connection of a replacement fluid container.

Aspect 9. The fluid circuit of Aspect 8 wherein the access site includes a replacement fluid connector.

Aspect 10. The fluid circuit of any one of Aspects 8 or 9 wherein the replacement fluid connector includes a luer or a spike.

Aspect 11. The fluid circuit of any one of Aspects 8 through 10 wherein an antimicrobial filter is positioned downstream of the access site.

Aspect 12. The fluid circuit of any one of Aspects 8 through 11 wherein the fluid replacement line further includes a drip chamber.

Aspect 13. The fluid circuit of Aspect 12 wherein the replacement fluid line includes a roller clamp positioned downstream of the drip chamber.

Aspect 14. The fluid circuit of Aspect 12 wherein the drip chamber is positioned downstream of the antimicrobial filter.

Aspect 15. A method for maintaining fluid volume in a donor during a manual collection of whole blood with a disposable fluid circuit of any one of Aspects 1 through 14 including: withdrawing blood form a donor through the donor access device and collecting a selected volume of blood in the primary container; closing the primary container line by actuating the flow controller on the primary container line; opening the replacement fluid line; and delivering a selected volume of replacement fluid from a replacement fluid container through the replacement fluid line to the donor through the access device.

Aspect 16. The method of Aspect 15 including delivering the replacement fluid to the donor prior to arriving at the selected volume of blood collected in the primary container.

Aspect 17. The method of Aspect 15 including delivering the replacement fluid to the donor after arriving at the selected volume of blood collection.

Aspect 18. The method of Aspect 17 further including permanently sealing the flow path of the primary container line.

Aspect 19. The method of any one of Aspects 15 through 18 including controlling the flow of the replacement fluid to the donor.

Aspect 20. The method of any one of Aspects 15 through 19 further including determining the volume of replacement fluid to be delivered to the donor.

Aspect 21. The method of Aspect 16 including pausing the withdrawing of blood form the donor prior to opening the replacement fluid line.

It will be understood that the embodiments described above are illustrative of some of the applications of the principles of at the present subject matter. Numerous modifications may be made by those skilled in the art without departing from the spirit and scope of the subject matter disclosed herein, including those combinations of features that are individually disclosed or claimed herein. For the reasons, the scope hereof is not limited to the above description.

1. A disposable fluid circuit for the manual collection of whole blood, comprising:
a primary container for collecting and processing whole blood and one or more secondary containers downstream of and in openable flow communication with said primary container;
a donor access device and a donor access line defining a flow path in flow communication with said donor access device;
a replacement fluid container containing a replacement fluid; and
a branch member in flow communication with said donor access line, said branch member including a first branch in openable flow communication with said primary container through a primary container line and a second branch in openable flow communication with said replacement fluid container by a replacement fluid line.

2. The fluid circuit of claim 1, wherein the replacement fluid line comprises an openable cannula between said second branch and said replacement fluid container.

3. The fluid circuit of claim 1, wherein said replacement fluid line further comprises a drip chamber positioned downstream of the cannula.

4. The fluid circuit of claim 1, wherein said replacement line further comprises a flow controller on said replacement fluid line.

5. The fluid circuit of claim 4, wherein said flow controller comprises a reopenable clamp.

6. The fluid circuit of claim 1, wherein said primary container line includes a re-openable flow controller.

7. The fluid circuit of claim 1, wherein the replacement fluid is saline, or a crystalloid or colloid solution.

8. A disposable fluid circuit for the manual collection of whole blood comprising:
a primary container for collecting and processing whole blood and one or more secondary containers in openable flow communication with said primary container; a donor access device and a donor access line defining a flow path in flow communication with said donor access device;
a branch member in flow communication with said donor access line, said branch member including a first branch in openable flow communication with said primary container through a primary container line and a second branch in openable flow communication with a replacement fluid line, said replacement fluid line terminating in an access site for connection of a replacement fluid container.

9. The fluid circuit of claim 8, wherein the access site comprises a replacement fluid connector.

10. The fluid circuit of claim 8, wherein the replacement fluid connector comprises a luer or a spike.

11. The fluid circuit of claim 8, wherein an antimicrobial filter is positioned downstream of the access site.

12. The fluid circuit of claim 8, wherein said fluid replacement line further comprises a drip chamber.

13. The fluid circuit of claim 12, wherein the replacement fluid line comprises a roller clamp positioned downstream of the drip chamber.

14. The fluid circuit of claim 12, wherein said drip chamber is positioned downstream of said antimicrobial filter.

15. A method for maintaining fluid volume in a donor during a manual collection of whole blood with a disposable fluid circuit of claim 1, said method comprising:
withdrawing blood from a donor through the donor access device and collecting a selected volume of said blood in said primary container;
closing the primary container line by actuating said flow controller on said primary container line;
opening the replacement fluid line; and
delivering a selected volume of replacement fluid from a replacement fluid container through said replacement fluid line to said donor through said access device.

16. The method of claim 15, comprising delivering the replacement fluid to said donor prior to arriving at said selected volume of blood collected in said primary container.

17. The method of claim 15, comprising delivering said replacement fluid to said donor after arriving at said selected volume and said blood collection is collected.

18. The method of claim 17 further comprising permanently sealing said flow path of said primary container line.

19. The method of claim 15 comprising controlling the flow of said replacement fluid to said donor.

20. The method of claim 15 further comprising determining the volume of replacement fluid to be delivered to said donor.

21. The method of claim 16 comprising pausing said withdrawing of said blood from the donor prior to opening said replacement fluid line.
